(19) United States
(12) Patent Application Publication
(10) Pub. No.: US 2024/0231117 A1
(43) Pub. Date: Jul. 11, 2024

(54) OPTICAL DISC UNIT FOR ULTRAVIOLET STERILIZER, METHOD OF MANUFACTURING THE SAME, AND ULTRAVIOLET STERILIZER INCLUDING THE SAME

(71) Applicants: Hyundai Motor Company, Seoul (KR); Kia Corporation, Seoul (KR); HATBIT ILLUCOM CO., LTD, Bucheon-si (KR)

(72) Inventors: Han-Joo JANG, Hwaseong-Si (KR); Woo-Young CHOI, Seoul (KR); Jin-Won LEE, Suwon-Si (KR); Sang-Ki LEE, Hwaseong-Si (KR); Hyeon-Seok LEE, Hwaseong-Si (KR); Hong-Sik CHANG, Hwaseong-Si (KR); Kwan-Woo LEE, Suwon-Si (KR); Tae-Yoon KIM, Suwon-Si (KR); Young-Ho CHA, Incheon (KR); Jae-Gyun SA, Goyang-Si (KR); Heon-Cheol KIM, Goyang-Si (KR)

(73) Assignees: Hyundai Motor Company, Seoul (KR); Kia Corporation, Seoul (KR); HATBIT ILLUCOM CO., LTD, Bucheon-si (KR)

(21) Appl. No.: 18/204,199

(22) Filed: May 31, 2023

(30) Foreign Application Priority Data
Jan. 9, 2023 (KR) 10-2023-0003089

(51) Int. Cl. G02B 27/09; A61L 2/10; A61L 2/26; G02B 5/08; G11B 23/50

(52) U.S. Cl. CPC G02B 27/0977; A61L 2/10; A61L 2/26; G02B 5/0891; G02B 27/0916; G02B 27/0927; G11B 23/505; A61L 2202/11

(57) ABSTRACT

An optical disc unit for an ultraviolet sterilizer, which is applied to the ultraviolet sterilizer, includes an ultraviolet light emitting diode (LED) to reflect light irradiated from the ultraviolet LED, including a body portion including a hollow and a primary optical unit fixedly coupled to an internal diameter surface in the hollow of the body portion and disposed in a direction perpendicular to an emission surface of the ultraviolet LED to reflect light irradiated from the ultraviolet LED, and according to an exemplary embodiment of the present disclosure, it is possible to enhance a sterilization ability by increasing a beam angle of light irradiated from the ultraviolet LED and uniformly irradiating the light and also reduce a risk of light leakage to the outside in a space to which the ultraviolet LED is applied.

### CROSS-REFERENCE TO RELATED APPLICATION

The present application claims priority to Korean Patent Application No. 10-2023-0003089, filed on Jan. 9, 2023, the entire contents of which is incorporated herein for all purposes by this reference.

### BACKGROUND OF THE PRESENT DISCLOSURE

### Field of the Present Disclosure

The present disclosure relates to an optics for expanding a beam range of ultraviolet rays from a light source of an ultraviolet sterilizer, a sterilizer including the same, and a method of manufacturing the same.

### Description of Related Art

Ultraviolet sterilizers are provided and used in an enclosed space such as a console box of a vehicle using an ultraviolet light emitting diode (LED) for emitting light in an ultraviolet wavelength region having a sterilization function. However, it is difficult to expect a satisfactory level of sterilization effect only using the ultraviolet LED.

Furthermore, the light from the ultraviolet LED passes through a lens, and the light is refracted through the lens so that the ultraviolet rays spread widely in an internal space, and the present disclosure relates to an optics for the present role.

In the conventional art related to the lens, the ultraviolet transmission function protrusion-shaped TIR refractive lens member is applied so that the ultraviolet ray emission function is not permanently impaired by forming the protrusion-shaped total internal reflection (TIR) refractive lens shape by a method, such as processing/polishing, using a material called fused silica glass (also known as a quartz glass) known to transmit ultraviolet rays best, and the ultraviolet transmission function protrusion-shaped TIR refractive lens member is directly attached to the upper end surface of the LED package body (housing) by bonding, etc.

In these related art, the ultraviolet ray irradiated from the inside of the ultraviolet LED package is finally irradiated (emitted) by transmitting the inside of the transparent body of the protrusion-shaped (dome-shaped) TIR refractive lens member rather than the flat plate shape (plate-shaped) lens member.

However, although the TIR refractive lenses are advanced from the flat plat shape to the protrusion-shaped (dome-shaped), the beam angle of ultimately irradiated ultraviolet ray is still limited.

Furthermore, because raw materials and processing costs are expensive, there is a limit to application to ultraviolet sterilizers.

In addition to the dome shape, there are technologies for various other lens shapes, but even with such a diversification of the shape, the beam angle of the irradiated ultraviolet ray is not greatly improved, and the TIR refractive lens is unfavorable in terms of the manufacturing price due to raw materials and processing.

FIG. 1 shows a beam angle of a general ultraviolet ray, and the above-described related arts also have the beam angle.

As shown, all types of ultraviolet (including visible) LED directional lamps emit cone-shaped light energy. As shown, an intensity of the light energy emitted from the LED is greatest at the center of a cone, and the light energy is gradually reduced toward an edge portion of the cone.

The intensity of the light energy in a cone-shaped light energy region is concentrated at the center of the LED up to 50%, the present portion of the entire cone of light is generally called the beam angle, and a range exceeding 50% becomes a spill region in which the intensity of light energy is very weak.

This does not mean that a 100% sterilization (disinfection) power is guaranteed against all bacteria to be sterilized even in a range of the beam angle.

FIG. 2 is an example of light distribution inside a console box 1 by an ultraviolet LED 3 when an ultraviolet sterilizer 2 is provided on an internal upper end portion of the console box 1.

As shown, it may be seen that the light energy is very weak in the spill region which is out of the beam angle, and thus the sterilization ability is inevitably low.

However, increasing the number of LEDs is unfavorable in terms of the manufacturing cost.

Meanwhile, because the ultraviolet sterilizer is provided on the internal upper end portion of the console box, the ultraviolet light may leak through a gap between the housing and an upper cover for opening and closing the console box.

There occurs a phenomenon that the ultraviolet light leaks from a sterilization tray and destroys the DNA in the cells of living organisms, causing dead cells to be separated from the skin tissue, and the dead cells have physiochemical energy capable of destroying the DNA of the living organisms and killing the cells of the living organisms.

The information included in this Background of the present disclosure is only for enhancement of understanding of the general background of the present disclosure and may not be taken as an acknowledgement or any form of suggestion that this information forms the prior art already known to a person skilled in the art.

### BRIEF SUMMARY

Various aspects of the present disclosure are directed to providing an optical disc unit for an ultraviolet sterilizer, which can enhance a sterilization ability by increasing a beam angle of light irradiated from an ultraviolet light emitting diode (LED) and uniformly irradiating the light and also reducing a risk of light leakage to the outside in a space to which the ultraviolet sterilizer is applied, a method of manufacturing the same, and an ultraviolet sterilizer including the same.

An optical disc unit for an ultraviolet sterilizer according to one aspect of the present disclosure, which is applied to the ultraviolet sterilizer including an ultraviolet light emitting diode (LED) to reflect light irradiated from the ultraviolet LED, includes a body portion including a hollow and a primary optical unit fixedly coupled to an internal diameter surface in the hollow of the body portion and disposed in a direction perpendicular to an emission surface of the ultraviolet LED to reflect the light irradiated from the ultraviolet LED therethrough.

Furthermore, the primary optical unit may include a first reflection portion disposed adjacent to the ultraviolet LED, wherein a length of an external circumference in the first reflection portion increases upwards from the ultraviolet LED in a direction in which the light is irradiated and a second reflection portion extending from the first reflection portion, wherein a length of an external circumference in the second reflection portion reduces upwards from the first reflection portion.

Furthermore, the first reflection portion and the second reflection portion may have a shape of cone or a shape of a polygonal pyramid.

Furthermore, the internal diameter surface of the body portion may include a first internal diameter surface perpendicular to the emission surface and a second internal diameter surface extending from the first internal diameter surface and formed to be inclined with a predetermined angle in a direction of an external diameter surface of the body portion in an upward direction of the body portion.

Furthermore, an interface between the first reflection portion and the second reflection portion may be formed upwards from an interface between the first internal diameter surface and the second internal diameter surface with respect to the direction in which light is irradiated from the ultraviolet LED.

Therefore, the light irradiated from the ultraviolet LED and reflected by the first reflection portion may be reflected by the second internal diameter surface.

Meanwhile, a material of the optical disc unit may be any one of polycarbonate (PC), nylon, and an Al metal material.

Next, an ultraviolet sterilizer according to one aspect of the present disclosure includes the optical disc unit, a substrate provided in a housing and on which the optical disc unit is mounted, and an ultraviolet LED mounted on the substrate and disposed in the hollow of the optical disc unit.

Furthermore, an interface between the first internal diameter surface and the second internal diameter surface may be formed upwards from the ultraviolet LED with respect to a direction in which the light from the ultraviolet LED is irradiated.

Furthermore, an interface between the first reflection portion and the second reflection portion may be formed upwards from the interface between the first internal diameter surface and the second internal diameter surface with respect to the direction in which the light from the ultraviolet LED is irradiated.

Therefore, the light irradiated from the ultraviolet LED and reflected by the first reflection portion may be reflected by the second internal diameter surface.

Next, a method of manufacturing an optical disc unit for an ultraviolet sterilizer according to one aspect of the present disclosure includes injecting the optical disc unit with Polycarbonate (PC) or nylon material and depositing aluminum (Al) on a surface of the injected optical disc unit.

Alternatively, the method includes die-casting the optical disc unit with an Al metal material and depositing Al on a surface of the die-casted optical disc unit.

According to the optical disc unit according to an exemplary embodiment of the present disclosure, it is possible to irradiate the ultraviolet energy in a form of high uniformity that maintains more detailed and precise uniformity by expanding the beam angle compared to the conventional refractive lenses using the transparent medium, more rapidly achieving the goal of sterilization within 10 minutes per irradiation, which is required by vehicle manufacturers and customers.

Furthermore, it is possible to rapidly manufacture components of consistent quality by general manufacturing methods instead of the quartz glass raw material and machining and polishing processes.

Furthermore, it is possible to significantly reduce or lower the light leakage of the ultraviolet sterilization system in advance by significantly reducing the light leakage region, which is a disadvantage of the ultraviolet ray transmission type medium, which can become an advantage of greatly mitigating the risk level of the entire sterilization system.

The methods and apparatuses of the present disclosure have other features and advantages which will be apparent from or are set forth in more detail in the accompanying drawings, which are incorporated herein, and the following Detailed Description, which together serve to explain certain principles of the present disclosure.

### BRIEF DESCRIPTION OF THE DRAWINGS

FIG. 1 shows a beam angle of general ultraviolet ray.

FIG. 2 is an example of light distribution when the conventional ultraviolet sterilizer is provided in a console box.

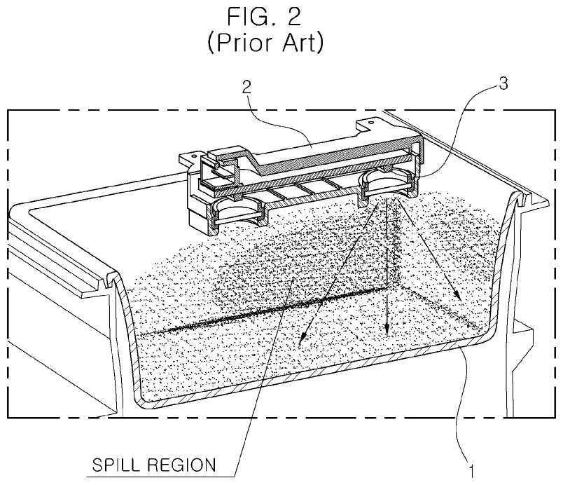

FIG. 3 shows an ultraviolet sterilizer to which an optical disc unit for an ultraviolet sterilizer according to an exemplary embodiment of the present disclosure is applied.

FIG. 4 shows the optical disc unit for the ultraviolet sterilizer according to an exemplary embodiment of the present disclosure.

FIG. 5 shows a beam angle by the optical disc unit for the ultraviolet sterilizer according to an exemplary embodiment of the present disclosure.

FIG. 6 is an example of light distribution when an ultraviolet sterilizer to which the optical disc unit for the ultraviolet sterilizer according to an exemplary embodiment of the present disclosure is applied is provided in a console box.

FIG. 7 is a flowchart for optimizing the optical disc unit for the ultraviolet sterilizer according to an exemplary embodiment of the present disclosure.

It may be understood that the appended drawings are not necessarily to scale, presenting a somewhat simplified representation of various features illustrative of the basic principles of the present disclosure. The predetermined design features of the present disclosure as included herein, including, for example, specific dimensions, orientations, locations, and shapes will be determined in part by the particularly intended application and use environment.

In the figures, reference numbers refer to the same or equivalent portions of the present disclosure throughout the several figures of the drawing.

### DETAILED DESCRIPTION

Reference will now be made in detail to various embodiments of the present disclosure(s), examples of which are illustrated in the accompanying drawings and described below. While the present disclosure(s) will be described in conjunction with exemplary embodiments of the present disclosure, it will be understood that the present description is not intended to limit the present disclosure(s) to those exemplary embodiments of the present disclosure. On the other hand, the present disclosure(s) is/are intended to cover not only the exemplary embodiments of the present disclosure, but also various alternatives, modifications, equivalents and other embodiments, which may be included within the spirit and scope of the present disclosure as defined by the appended claims.

To fully understand the present disclosure, the operational advantages of the present disclosure, and the objects achieved by the practice of the present disclosure, reference should be made to the accompanying drawings showing exemplary embodiments of the present disclosure and the contents described in the accompanying drawings.

In describing the exemplary embodiments of the present disclosure, known technologies or repetitive descriptions which may unnecessarily obscure the subject matter of the present disclosure will be reduced or omitted.

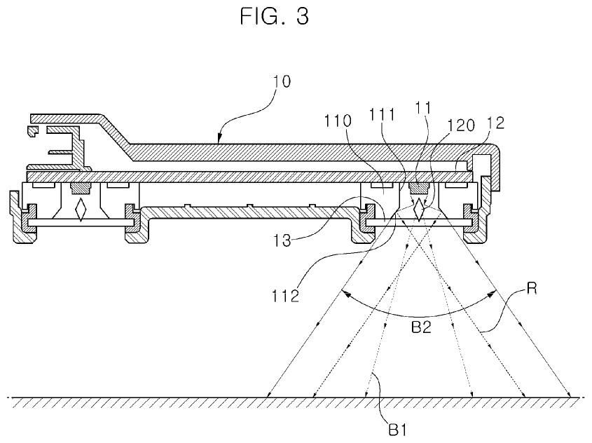

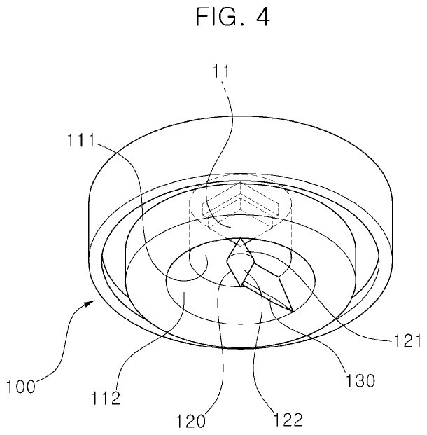

FIG. 3 shows an ultraviolet sterilizer to which an optical disc unit for an ultraviolet sterilizer according to an exemplary embodiment of the present disclosure is applied, and FIG. 4 shows the optical disc unit for the ultraviolet sterilizer according to an exemplary embodiment of the present disclosure.

Hereinafter, the optical disc unit for the ultraviolet sterilizer according to an exemplary embodiment of the present disclosure and the ultraviolet sterilizer including the same will be described with reference to FIG. 3 and FIG. 4.

The present disclosure is to improve the sterilization performance of an ultraviolet sterilizer 10 provided in an enclosed space such as a console box 20 of a vehicle and prevent light leakage to the outside.

In the ultraviolet sterilizer 10, an ultraviolet light emitting diode (LED) 11 is mounted on a substrate 12, an opening is formed in a direction in which light from the ultraviolet LED 11 of a housing is irradiated, and a cover 13 for covering the opening is mounted.

As shown, the light irradiated from the ultraviolet LED 11 has a cone-shaped beam range, and B1 corresponds to an angle to a region in which light energy reaches 50% in a direction perpendicular to the ultraviolet LED 11, which corresponds to a region in which the conventional beam angle, that is, light energy is concentrated,

The present disclosure is to expand the beam angle to an angle indicated by B2 so that light may be uniformly irradiated to the entire beam range of the ultraviolet LED 11.

To the present end, an optical disc unit 100 according to an exemplary embodiment of the present disclosure is mounted on the substrate 12 to surround the ultraviolet LED 11 so that the ultraviolet LED 11 is disposed on a center portion of the optical disc unit 100.

The optical disc unit 100 includes a body portion 110 and a primary optical unit 120, and the body portion 110 is mounted on the substrate 12 to surround the ultraviolet LED 11 to form an exterior.

The body portion 110 has a donut shape in which a hollow is formed, and the ultraviolet LED 11 is disposed in the hollow.

An internal diameter surface of the body portion 110 may be formed to be divided into a first internal diameter surface 111 and a second internal diameter surface 112, and the first internal diameter surface 111 corresponds to a circular cross section perpendicular to a lower surface (corresponding to an upper surface in the drawing) of the body portion 110.

The second internal diameter surface 112 extends from the first internal diameter surface 111 and is formed to be inclined in a direction of an external diameter surface of the body portion 110 toward the upper surface (corresponding to the lower surface in the drawing) of the body portion 110.

As shown, an interface between the first internal diameter surface 111 and the second internal diameter surface 112 is formed in an upward direction of the ultraviolet LED 11 with respect to the direction in which light is irradiated.

Therefore, the second internal diameter surface is configured as a secondary optics for reflecting the light irradiated from the ultraviolet LED 11, and the primary optical unit 120 to be described below is configured as a primary optics.

As shown, the cover 13 may be coupled to be in contact with the upper surface of the body portion 110.

Next, the primary optical unit 120 may be spaced in the upward direction (upward direction and downward direction are defined with respect to the direction in which light is irradiated) of the ultraviolet LED 11 in a direction perpendicular to an emission surface of the ultraviolet LED 11 and fixed to the first internal diameter surface 111 of the body portion 110 by a separate fixing portion 130.

The primary optical unit 120 is configured as the primary optics for primarily reflecting the light irradiated from the ultraviolet LED 11 and expands the beam angle to the angle indicated by B2 so that the light may be uniformly irradiated to the entire beam range of the ultraviolet LED 11.

To the present end, the primary optical unit 120 includes a first reflection portion 121 close to the ultraviolet LED 11 and including a shape in which a length of an external circumference increases upward and a second reflection portion 122 including a shape in which a length of an external circumference reduces upwards from the first reflection portion 121.

The first reflection portion 121 and the second reflection portion 122 may have a shape of cone or a shape of a polygonal pyramid and have a shape sharing a bottom surface with each other, that is, a bi-conical shape or a bi-polygonal pyramid shape as shown.

Therefore, as shown, the light irradiated from the ultraviolet LED 11 is reflected by an inclined surface of the first reflection portion 121 and irradiated to the second internal diameter surface 112 so that the light may be irradiated in a linear direction including the greatest range B2 of the beam angle by the inclined surface of the second internal diameter surface 112 as shown and in a direction R indicated by the dotted line between B1 and B2, which is the conventional beam angle.

According to an exemplary embodiment of the present disclosure, the light may be uniformly irradiated to the conventional Spill region by a combination of the primary optical unit 120 which is the primary optics and the second internal diameter surface 112 which is the secondary optics to expand the beam angle.

For the present structure, an interface corresponding to bottom surfaces of the first reflection portion 121 and the second reflection portion 122 is formed upwards from the interface between the first internal diameter surface 111 and the second internal diameter surface 112 with respect to the direction in which light is irradiated.

Furthermore, angles of the inclined surfaces of the first reflection portion 121 and the second reflection portion 122 may be set according to the setting of a desired beam angle, and as described above, according to an exemplary embodiment of the present disclosure, the beam angle may be adjusted as a wide angle or a narrow angle by setting a height of the primary optical unit 120, the angles of the inclined surfaces of the primary optical unit 120, a separation distance between the first internal diameter surface 111 and the primary optical unit 120, and an angle of the inclined surface of the second internal diameter surface 112.

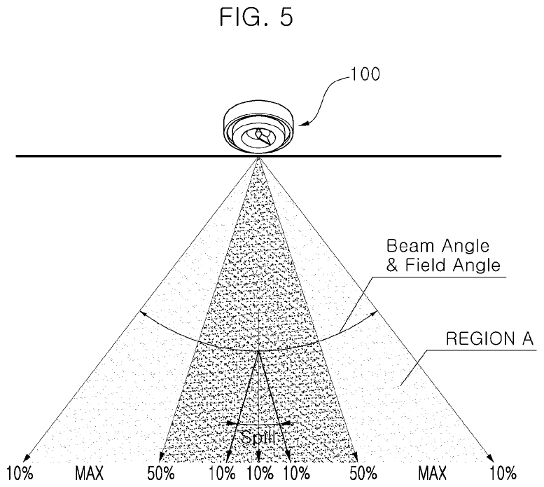

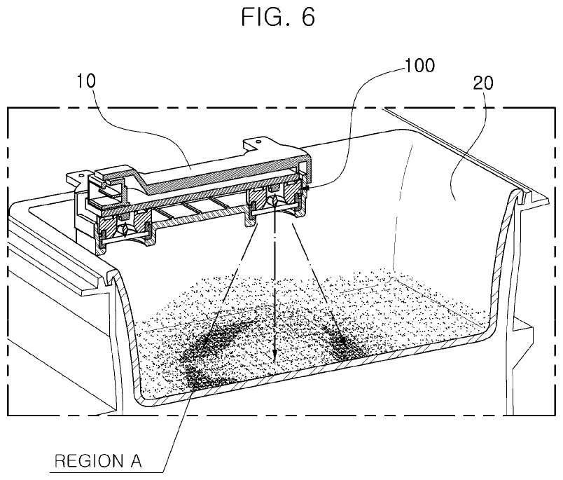

Next, FIG. 5 shows a beam angle by the optical disc unit for the ultraviolet sterilizer according to an exemplary embodiment of the present disclosure, and FIG. 6 is an example of light distribution when an ultraviolet sterilizer to which the optical disc unit for the ultraviolet sterilizer according to an exemplary embodiment of the present disclosure is applied is provided in a console box.

As shown, all kinds of ultraviolet (including visible) LED directional lamps emit cone-shaped light energy, and an intensity of the light energy emitted from the LED is greatest at the center portion of the cone, and the light energy is gradually reduced toward an edge portion of the cone.

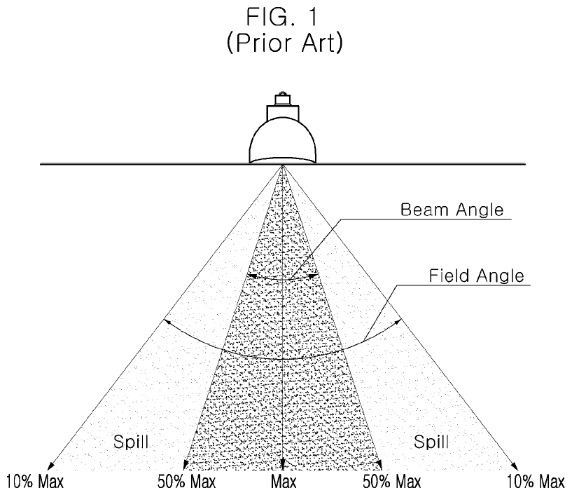

Furthermore, as in the related art described in FIG. 1, the intensity of the light energy emitted from the LED using the protrusion-shaped TIR refractive lens or the flat plate-shaped lens member is greatest at the center portion of the cone, and the light energy is gradually reduced toward the edge portion of the cone.

However, as shown in FIG. 5 and FIG. 6, it may be seen that when the optical disc unit according to an exemplary embodiment of the present disclosure is applied, the intensity of the light energy emitted from the ultraviolet LED is weakest at the center portion of the cone and becomes the maximum between the edge portion of the cone and the 50% interface (region A).

The optical disc unit 100 according to an exemplary embodiment of the present disclosure may be manufactured by injection+Al high reflection deposition or die-casting+Al high reflection deposition using polycarbonate (PC), nylon, or an Al metal material.

Therefore, with the above-described characteristics, the primary optical unit 120 including the reflection and refraction shape, which is the primary optics, is formed just under a center portion of an LED adjacent to field to reflect and refract a hot spot on a center portion of the ultraviolet LED light energy, which is the representative characteristic of Lambertian optical characteristics, to the second internal diameter surface 112, which is the secondary optics, and branch the hot spot to 360 degrees, mitigating the hot spot on the center portion.

In other words, it is possible to adjust the beam angle of the final light energy reflected from the primary optics and incident on a reflection-shaped surface of the secondary optics of a side surface portion of the LED near field so that the light energy may be uniformly irradiated to a beam portion, which is a sterilization region.

Therefore, it is possible to adjust the beam angle to the wide angle and the narrow angle and uniformize the ultraviolet beam region as wide as 15% or more.

Furthermore, because an energy reduction region corresponding to the beam angle of 50% may be formed at the center portion of a beam angle of a light intensity distribution (cone shape) to minimize the energy reduction region (area), unnecessary light leakage may be blocked (cutoff function).

Furthermore, raw materials and processing costs are very cheap compared to the quartz glass, and mass production is possible rapidly with regular quality.

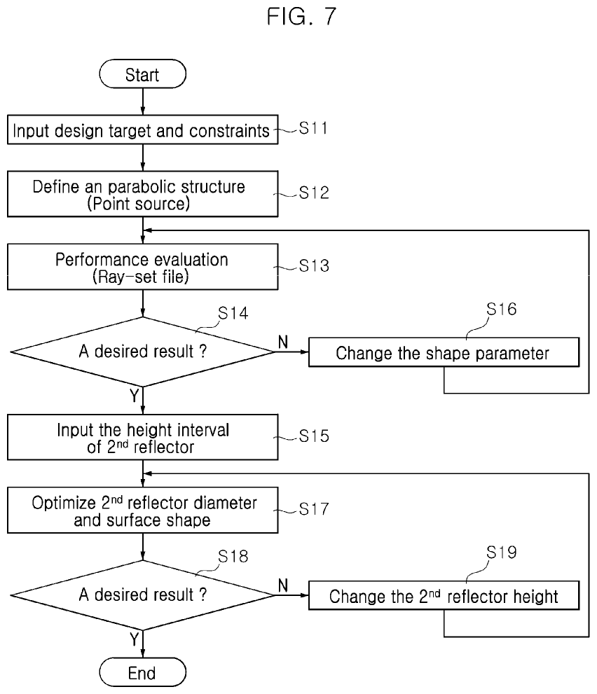

Next, FIG. 7 is a flowchart for optimizing the optical disc unit for the ultraviolet sterilizer according to an exemplary embodiment of the present disclosure.

The conventional quartz glass TIR type refractive lens has a structure including a parabolic total reflection surface for causing the TIR and a free-shape refraction surface and can obtain relatively high luminous flux efficiency even at a luminous flux angle of less than 10 degrees. However, it is known that the luminous flux efficiency is rapidly reduced to 50% or less when a size of the light source is about 15% or more of the total diameter of the lens.

Furthermore, as a size or a volume of the TIR refractive lens increases to maximize efficiency (to improve uniformity), an absorption loss by the lens medium increases rapidly, and thus it is difficult to apply the TIR refractive lens to the primary optics and the secondary optics, which use the ultraviolet LED package as the light source.

The design requirements of the three TIR refractive lenses may not be implemented by the cutting and polishing methods using the quartz glass material.

Meanwhile, it is possible to enhance the luminous flux efficiency using the new optical disc technology in which the reflection optics is provided in the primary/secondary near fields without not only the effect of the absorption loss due to the quartz glass medium caused by not using the TIR refractive lens optical technology is not affected but also the effect of the thickness of the medium.

However, when only a disc reflector, which is the primary optics, is used, there may be beams that proceed directly from the light source to an illumination surface without being reflected by the reflector.

Therefore, concentric leaked light, so-called a satellite ring, is generated, degrading the uniformity of ultraviolet ray illumination.

Therefore, the relationship between the luminous flux angle and the size of the optics, which are set as the design goal of not only optimizing the reflection angle and refraction angle of the disc reflector which is the primary optics but also optimizing the original shape of the reflection surface which is the secondary optics and the design criteria for preventing the satellite ring are introduced, and the illumination results in which the uniformity for various design (wide angle-narrow angle) conditions is secured were predicted.

In the optimally designed secondary optics, the luminous flux efficiency of 79.6% could be obtained at the luminous flux angle of 15.4 degrees, and the Gaussian distribution similarity of the illuminance distribution was 98.5%, and thus most of the satellite rings could be suppressed.

Compared to the provided secondary optics, it is also possible to implement narrow-angle illumination within 10 degrees, but in the instant case, it was confirmed that the characteristics such as luminous flux efficiency and illuminance distribution are degraded, and the present trade-off relationship will be able to be applied to implement a specific illumination purpose. In the future, research will be conducted on a secondary optics including various configurations, such as a reflection-refraction composite optics including a reflector and a lens as well as a double reflector structure.

Furthermore, the primary and secondary reflection optics using the ultraviolet LED package need to satisfy the pursuit of high luminous flux efficiency and a constant luminous flux angle according to the beam angle and need to have a luminous intensity distribution in a form of a Gaussian function.

In the design of the optical disc unit according to an exemplary embodiment of the present disclosure, the above-described design target, that is, the beam angle and the constraints are set (S11), the parabolic structure is defined (S12), and performance is evaluated (S13).

As a result of the performance evaluation (S13) by the structure defined by S12, it is confirmed whether the desired result in S11 is satisfied (S14).

When the desired result is not satisfied, the above-described shape parameters are changed (S16) and S13 is re-performed.

When it is determined that the desired result is satisfied, a height interval of a 2nd reflector is input (S15), and a diameter and a surface shape of the 2nd reflector are optimized (S17).

Later, whether the desired result is satisfied is re-checked (S18), and when the desired result is not satisfied, the height of the 2nd reflector is changed (S19), and S17 is re-performed.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The foregoing descriptions of specific exemplary embodiments of the present disclosure have been presented for purposes of illustration and description. They are not intended to be exhaustive or to limit the present disclosure to the precise forms disclosed, and obviously many modifications and variations are possible in light of the above teachings. The exemplary embodiments were chosen and described in order to explain certain principles of the invention and their practical application, to enable others skilled in the art to make and utilize various exemplary embodiments of the present disclosure, as well as various alternatives and modifications thereof. It is intended that the scope of the present disclosure be defined by the Claims appended hereto and their equivalents.

What is claimed is:

1. An optical disc unit for an ultraviolet sterilizer, which is applied to the ultraviolet sterilizer including an ultraviolet light emitting diode (LED) to reflect light irradiated from the ultraviolet LED, the optical disc unit comprising:
a body portion including a hollow; and
a primary optical unit fixedly coupled to an internal diameter surface in the hollow of the body portion and disposed in a direction perpendicular to an emission surface of the ultraviolet LED to reflect the light irradiated from the ultraviolet LED therethrough.

2. The optical disc unit of claim 1, wherein the primary optical unit includes:
a first reflection portion disposed adjacent to the ultraviolet LED, wherein a length of an external circumference in the first reflection portion increases upwards from the ultraviolet LED in a direction in which the light is irradiated; and
a second reflection portion extending from the first reflection portion, wherein a length of an external circumference in the second reflection portion reduces upwards from the first reflection portion.

3. The optical disc unit of claim 2, wherein the first reflection portion and the second reflection portion include a shape of cone.

4. The optical disc unit of claim 2, wherein the first reflection portion and the second reflection portion include a shape of a polygonal pyramid.

5. The optical disc unit of claim 2, wherein the internal diameter surface of the body portion includes:
a first internal diameter surface perpendicular to the emission surface; and
a second internal diameter surface extending from the first internal diameter surface and formed to be inclined with a predetermined angle in a direction of an external diameter surface of the body portion along an upward direction of the body portion.

6. The optical disc unit of claim 5, wherein an interface between the first reflection portion and the second reflection portion is formed upwards from an interface between the first internal diameter surface and the second internal diameter surface with respect to the direction in which light is irradiated from the ultraviolet LED.

7. The optical disc unit of claim 6, wherein the light irradiated from the ultraviolet LED and reflected by the first reflection portion is reflected by the second internal diameter surface.

8. The optical disc unit of claim 5, wherein a material of the optical disc unit is one of polycarbonate (PC), nylon, and an Al metal material.

9. An ultraviolet sterilizer comprising:
the optical disc unit of claim 5;
a housing;
a substrate provided in the housing and on which the optical disc unit is mounted; and
the ultraviolet LED mounted on the substrate and disposed in the hollow of the optical disc unit.

10. The ultraviolet sterilizer of claim 9, wherein an interface between the first internal diameter surface and the second internal diameter surface is formed upwards from the ultraviolet LED with respect to a direction in which the light from the ultraviolet LED is irradiated.

11. The ultraviolet sterilizer of claim 10, wherein an interface between the first reflection portion and the second reflection portion is formed upwards from the interface between the first internal diameter surface and the second internal diameter surface with respect to the direction in which the light from the ultraviolet LED is irradiated.

12. The ultraviolet sterilizer of claim 11, wherein the light irradiated from the ultraviolet LED and reflected by the first reflection portion is reflected by the second internal diameter surface.

13. A method of manufacturing an optical disc unit for an ultraviolet sterilizer, the method comprising:
injecting the optical disc unit of claim 5 with Polycarbonate (PC) or nylon material or die-casting the optical disc unit of claim 5 with an Al metal material; and
depositing aluminum (Al) on a surface of the injected or die-casted optical disc unit.
